(19) United States
(12) Patent Application Publication
(10) Pub. No.: US 2010/0292765 A1
(43) Pub. Date: Nov. 18, 2010

(54) Apparatus and Method for Cooling the Brain Using a Liquid

(75) Inventor: Alex Etwil, Koebenhavn N. (DK)

Correspondence Address: BRINKS HOFER GILSON & LIONE/CHICAGO/COOK, PO BOX 10395, CHICAGO, IL, 60610, US

(73) Assignees: William Cook Europe ApS, DK-4632 (DK); Cook Incorporated, Bloomington, IN (US)

(21) Appl. No.: 12/524,342

(22) PCT Filed: Jan. 28, 2008
(86) PCT No.: PCT/US08/01096

§ 371 Date: Jun. 28, 2010

Related U.S. Application Data
(60) Provisional application No. 60/897,744, filed on Jan. 26, 2007.

(51) Int. Cl. A61F 7/12

(52) U.S. Cl. 607/105

(57) ABSTRACT

Apparatus for cooling the brain comprises a pernasal intubation tube for insertion through one of the nostrils of a patient requiring brain cooling; an inflatable occluder provided the tube for inflation to occlude the nasopharynx of the patient behind the soft palate of the patient thereby to prevent passage of liquid between the nasal cavities and the mouth of the patient; a nostril closure arranged to cooperate with the pernasal intubation tube; an orifice for introduction of a liquid coolant into the nasal cavities; and a liquid vent for insertion in the other nostril of the patient for controlling venting of liquid coolant from the nasal cavities. Also described herein is a method of cooling the brain of a patient in danger of suffering brain damage comprising occluding the air passage leading from the nasal cavities to the mouth cavity of the patient behind the soft palate of the patient and irrigating the nasal cavities of the patient with a coolant liquid.

### TECHNICAL FIELD

This invention relates to an apparatus and method for cooling the brain.

### BACKGROUND OF THE INVENTION

This invention relates to an apparatus and method for cooling the brain.

Lowering body temperature is thought to lessen the damaging effects of stroke.

There is a need in the art for an improved brain cooling apparatus which is simple and fast to use in emergency when a patient is in danger of suffering brain damage, for example, following a stroke. It is also desirable to provide a brain cooling apparatus which can be reliably and speedily put into use without significant difficulty by paramedics or nurses in a First Aid situation without requiring intervention by a physician or surgeon.

In addition, there is a need for an improved method of effecting brain cooling in a patient who has suffered a stroke or is otherwise in danger of undergoing brain damage unless speedy medical intervention is applied. In particular, there is a need for a method of effecting controlled cooling of the brain which can be put into operation simply and without the need for complex apparatus or for undertaking difficult medical procedures.

### SUMMARY OF THE INVENTION

The invention provides in one aspect an apparatus for cooling the brain comprising:

a pernasal intubator comprising a tube having a proximal portion, a distal end for insertion through one of the nostrils of a patient requiring brain cooling, and a first lumen extending from the proximal portion to a closed distal lumen end;

an inflatable occluder provided on a distal portion of the tube for inflation to occlude the nasopharynx of the patient behind the soft palate of the patient thereby to prevent passage of liquid between the nasal cavities and the mouth of the patient;

a nostril closure arranged to cooperate with the tube for closing the one nostril of the patient and preventing fluid escaping from the nasal cavity with which the one nostril communicates;

at least one orifice in communication with the first lumen and disposed in a wall of the tube between the nostril closure and the inflatable occluder for introduction of a liquid coolant from the first lumen of the tube into the nasal cavities; and

a liquid vent for insertion in the other nostril of the patient for controlling venting of liquid coolant from the nasal cavities of the patient.

In another aspect the invention provides a method of cooling the brain of a patient in danger of suffering brain damage comprising occluding the air passage leading from the nasal cavities to the mouth cavity of the patient behind the soft palate of the patient and irrigating the nasal cavities of the patient with a coolant liquid. Such a method does not involve obstruction of the oropharynx or laryngopharynx so that the patient can continue to breathe through his or her mouth with or without the aid of a respirator.

Conveniently in such a method the coolant liquid is supplied through one of the patient's nostrils and discharged through the other nostril. The coolant liquid can be supplied intermittently or continuously.

Hence a particularly preferred method of cooling the brain of a patient suffering from a condition which could lead to brain damage comprises the steps of:

providing a pernasal intubator comprising a tube having a proximal portion, a distal end, a first lumen extending between the proximal portion and a closed distal lumen end, an inflatable occluder on a distal portion of the tube, and at least one orifice in communication with the first lumen and disposed in a wall of the tube between the nostril closure and the inflatable occluder;

introducing the distal end of the tube through one of the nostrils of the patient until the inflatable occluder lies in the nasopharynx behind the soft palate of the patient;

inflating the inflatable occluder so as to occlude the nasopharynx of the patient in a region behind the soft palate of the patient, thereby to prevent passage of liquid between the nasal cavities and the mouth of the patient;

inserting into the other nostril of the patient a liquid vent for controlling venting of liquid from the nasal cavities of the patient;

introducing a coolant liquid into the nasal cavities through the first lumen of the tube and the orifice so as to bathe the lining of the nasal cavities with coolant liquid; and

venting liquid from the nasal cavities of the patient through the liquid vent.

The invention further provides a kit for cooling the brain of a patient in danger of suffering brain damage comprising:

a pernasal intubator comprising a tube having a proximal portion, a distal end for insertion through one of the nostrils of a patient requiring brain cooling and a first lumen extending from the proximal portion to a closed distal lumen end;

an inflatable occluder provided on a distal portion of the tube for inflation to occlude the nasopharynx of the patient thereby to prevent passage of liquid between the nasal cavities and the mouth of the patient;

a nostril closure arranged to cooperate with the tube for closing the one nostril of the patient and preventing fluid escaping from the nasal cavity with which the one nostril communicates;

at least one orifice in communication with the first lumen and disposed in a wall of the tube between the nostril closure and the inflatable occluder for introduction of a liquid coolant from the first lumen of the pernasal intubation tube into the nasal cavities;

a liquid vent for insertion in the other nostril of the patient for controlling venting of liquid coolant from the nasal cavities of the patient;

a source of a cold biologically acceptable liquid; and

a store for storing the source of cold biologically acceptable liquid and for maintaining same at a temperature suitable for effecting cooling of a patient's brain.

### BRIEF DESCRIPTION OF THE DRAWINGS

In the accompanying semi-diagrammatic drawings:

FIG. 1 is a vertical section through a patient's right nostril showing a pernasal intubation tube provided in accordance with the invention in position with its soft cuff inflated;

FIG. 2 is a cross section on an enlarged scale of the pernasal intubation tube of FIG. 1 taken on the line II-II of FIG. 1; and

FIG. 3A shows a cross section through a pernasal intubation tube according to a further embodiment of the present invention, showing the two lumens within the intubation tube;

FIG. 3B shows a cross section through a pernasal intubation tube according to a still further embodiment of the present invention, having a different arrangement of lumens to the embodiment of FIG. 3A;

FIG. 3C is a cross section through the distal end of a pernasal intubation tube in accordance with yet a further embodiment of the invention, showing a detailed view of its soft cuff in an inflated state;

FIG. 3D is a cross section through the distal end of an intubation tube in accordance with a still further embodiment of the invention, showing a detailed view of its soft cuff in a deflated state;

FIG. 3E is a cross section through the intubation tube of FIG. 3B, displaying the soft cuff in an inflated state;

FIG. 3F is a cross section through an inflatable occluder according to a still further embodiment of the present invention, the occluder comprising two soft cuffs, which are shown in an inflated state;

FIG. 3G shows a cross section through an inflatable occluder according to yet another embodiment of the present invention, which is formed integrally with the intubation tube;

FIG. 4 shows an inflation bulb, which is used to inflate an inflatable occluder of apparatus according to an embodiment of the present invention;

FIG. 5 shows a saline drip attachment, which is used to inflate an inflatable occluder of apparatus according to another embodiment of the present invention;

FIG. 6 shows an adapter, which may be used to connect the inflation and cooling fluid sources to the intubation tube;

### DETAILED DESCRIPTION

Embodiments of the present invention may provide apparatus for cooling the brain of a patient who is at risk of suffering brain damage as a result of a trauma, such as a stroke, which can—for example—be used in a First Aid context by paramedics and other emergency service workers in situations where a person has suffered a trauma, such as a stroke, in the course of his or her normal occupations. Such apparatus may be used quickly and safely under emergency situations without risk of causing damage to the lower part of the pharynx.

Examples of the invention may also provide a method of effecting brain cooling which can be speedily put into effect and which causes efficient cooling of a patient's brain following a traumatic episode, such as a stroke.

There will now be described a preferred embodiment of an apparatus for cooling the brain in accordance with the invention, which is illustrated in the accompanying drawings. This embodiment comprises a pernasal intubator comprising a tube which has a proximal portion and a distal end for insertion through one of the nostrils of a patient requiring brain cooling, as well as a first lumen extending from the proximal portion to a closed distal lumen end. The tube is further provided with an inflatable occluder provided on a distal portion of the tube for inflation to occlude the nasopharynx of the patient behind the soft palate of the patient thereby to prevent passage of liquid between the nasal cavities and the mouth of the patient. Admission of fluid to the inflatable occluder may be provided so as to effect inflation thereof so as to cause occlusion of the nasopharynx of the patient behind the soft palate of the patient. The apparatus also comprises a nostril closure arranged to cooperate with the tube for closing the one nostril of the patient and preventing fluid escaping from the nasal cavity with which the one nostril communicates. In addition the apparatus comprises at least one orifice in communication with the first lumen and disposed in a wall of the pernasal intubation tube between the nostril closure means and the inflatable occluder for introduction of a liquid coolant from the first lumen of the pernasal intubation tube into the nasal cavities. It further comprises a liquid vent for insertion in the other nostril of the patient for controlling venting of liquid coolant from the nasal cavities of the patient.

Preferably the first lumen is provided at its proximal end for connection to a source of cold coolant liquid. Such connection can be adapted for connection to a diffusion bag containing, for example, a cold saline solution, preferably a cold isotonic saline solution.

The inflatable occluder conveniently comprises a first cuff secured in fluid tight fashion to a distal portion of the tube. Alternatively the inflatable occluder may comprise a balloon secured in fluid tight fashion to a distal portion of the tube. If desired, the tube may be provided with a subsidiary inflatable cuff positioned proximally upstream from the first cuff or balloon.

Preferred apparatus comprises a second lumen terminating at its distal end at an aperture within the inflatable occluder. Such a second lumen can be arranged to communicate with a side branch tube provided towards the proximal end of the tube. Moreover a fluid connection is preferably provided at the proximal end of the second lumen for connection to a source or generator of pressurised fluid. Such a fluid connection can be adapted for connection to an inflation bulb.

It is also preferred that the nostril closure comprises a plug having a bore for receipt of the tube.

The liquid vent conveniently comprises a plug for insertion in the other nostril having a bore providing a passage for liquid between the nasal cavity with which the other nostril communicates and a liquid outlet for controlling outflow of liquid from that nasal cavity. Such a liquid outlet can comprise a valve or a tap.

In another aspect the invention comprises a kit comprising such apparatus in combination with a source of a cold biologically acceptable liquid, for example, one or more diffusion bags filled with cold saline solution, and a store for storing the source of cold biologically acceptable liquid and for maintaining same at a temperature suitable for effecting cooling of a patient's brain, for example, a refrigerator or insulated storage box.

In addition, there will be described a method of cooling the brain of a patient in danger of suffering brain damage comprising occluding the air passage leading from the nasal cavities to the mouth cavity of the patient behind the soft palate of the patient and irrigating the nasal cavities of the patient with a coolant liquid. Such a method leaves the oropharynx and laryngopharynx substantially unobstructed.

In particular, there will be described a method of cooling the brain of a patient suffering from a condition which could lead to brain damage that comprises several steps. These steps comprise:

providing a pernasal intubator comprising a tube having a proximal portion, a distal end, a first lumen extending between the proximal portion and a closed distal lumen end, an inflatable occluder on a distal portion of the tube, and at least one orifice in communication with the first lumen and disposed in a wall of the tube between the nostril closure and the inflatable occluder;

introducing the distal end of the tube through one of the nostrils of the patient until the inflatable occluder lies in the nasopharynx behind the soft palate of the patient;

inflating the inflatable occluder so as to occlude the nasopharynx of the patient in a region behind the soft palate of the patient, thereby to prevent passage of liquid between the nasal cavities and the mouth of the patient;

inserting into the other nostril of the patient a liquid venting for controlling venting of liquid from the nasal cavities of the patient;

introducing a coolant liquid into the nasal cavities through the first lumen of the tube and the orifice so as to bathe the lining of the nasal cavities with coolant liquid; and

venting liquid from the nasal cavities of the patient through the liquid vent.

In such a method the step of introducing the liquid vent means can be performed before the step of inserting the pernasal intubator. Likewise the step of inflating the inflatable occluder can be performed before or after insertion of the liquid vent.

The tube is inserted through the patient's nostril until the inflatable occluder lies behind the patient's soft palate. In this way the oropharynx and laryngopharynx of the patient are left substantially unobstructed so that the patient can continue to breathe through the mouth either unaided or with the assistance of a respirator.

Inflation of the inflatable occluder may comprise supplying air under pressure through a second lumen in the pernasal intubation tube, for example, by an inflation bulb.

Conveniently the cooling liquid is supplied from a diffusion bag containing a charge of a cold biologically acceptable liquid, such as a cold saline solution, preferably a cold isotonic saline solution.

Liquid may be vented from the nasal cavities through a valve or through a tap. In a preferred method liquid is vented from the nasal cavities into a diffusion bag.

The coolant liquid is preferably supplied at a temperature of from about +4° C. to about +10° C., typically at a rate of from about 25 ml/min to about 1000 ml/min.

It will normally be preferred that the temperature of the brain is monitored and that the coolant liquid is introduced at a rate sufficient to maintain the temperature of the brain at between about 32° C. and about 35° C.

In the drawings the reference numerals indicate like parts throughout.

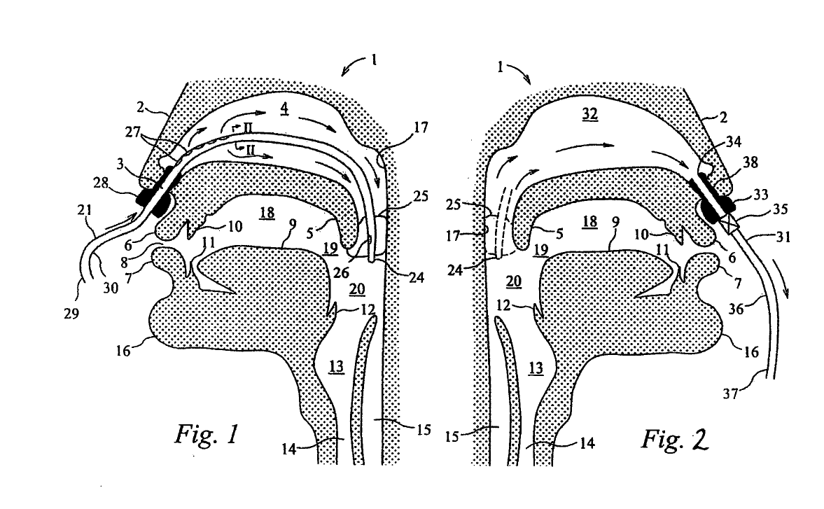

Referring to the drawings, FIG. 1 is a semi-diagrammatic cross section through part of a person's head 1, including the nose 2, right nostril 3, right nasal cavity 4, soft palate 5, lips 6 and 7, mouth 8, tongue 9, teeth 10 and 11, epiglottis 12, larynx 13, trachea 14, oesophagus 15, chin 16, nasopharynx 17, mouth cavity 18, oropharynx 19, and laryngopharynx 20.

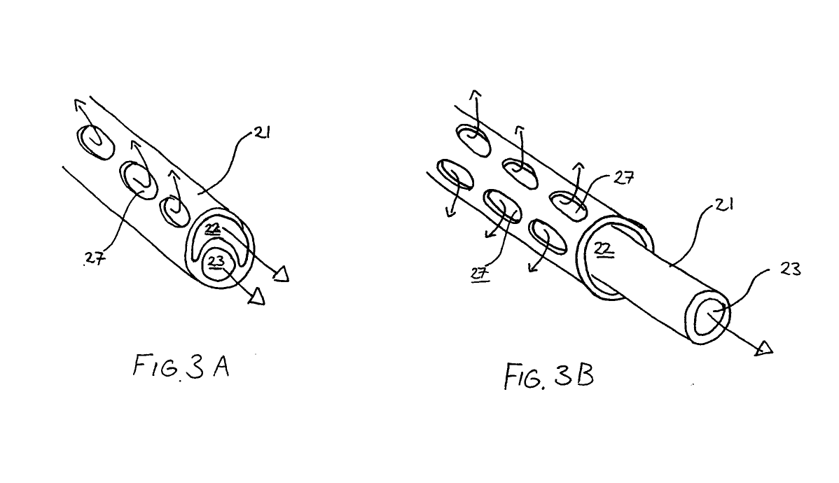

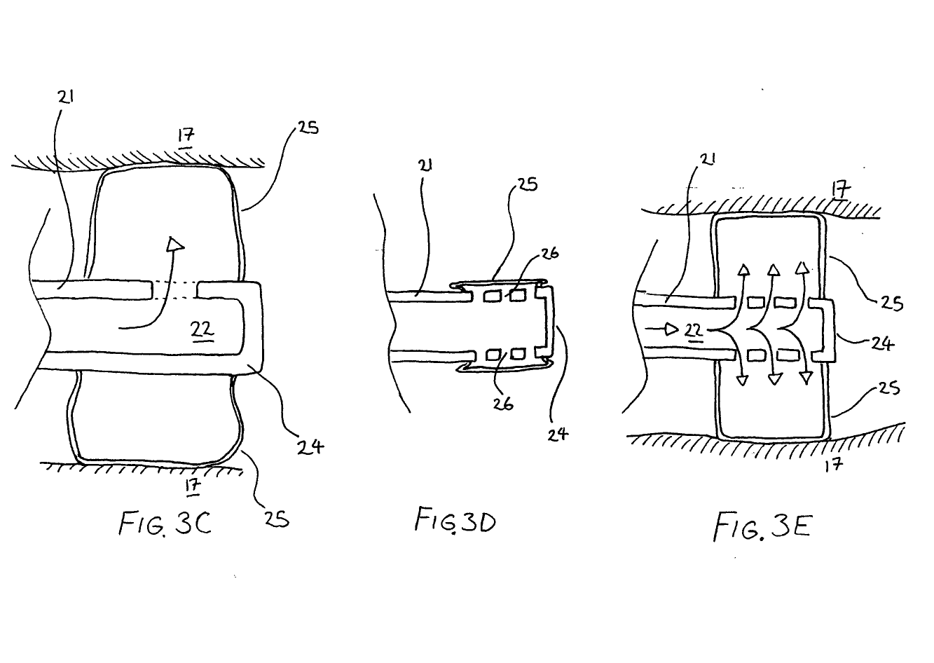

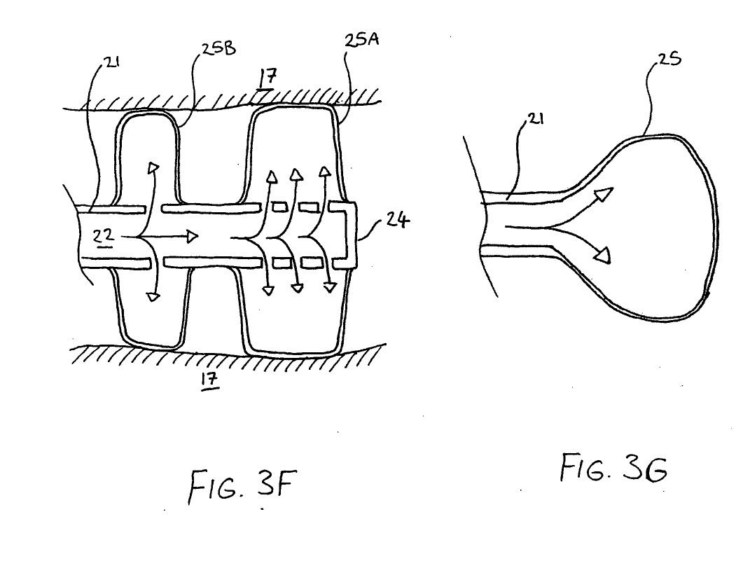

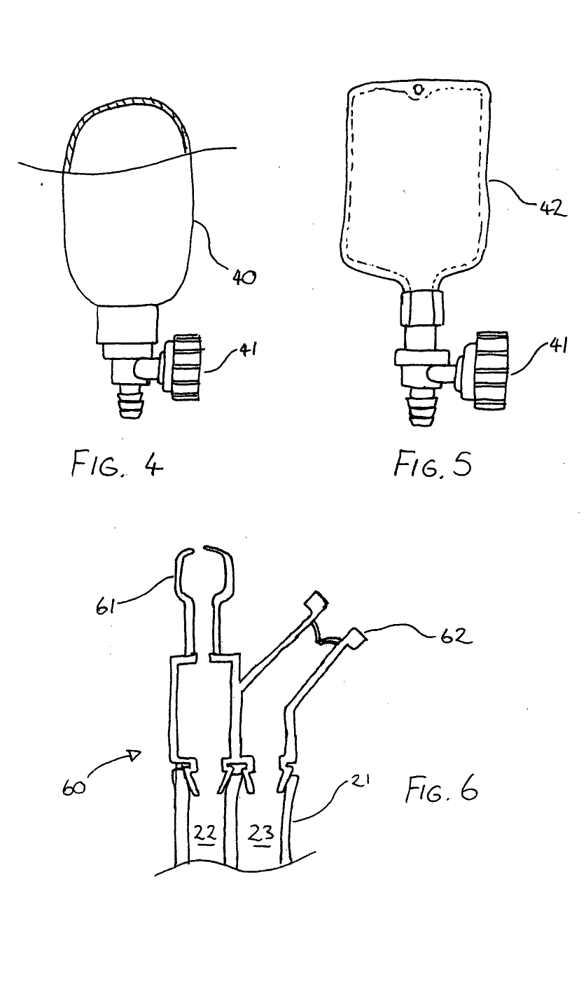

The illustrated apparatus includes a pernasal intubation tube 21 which has, as shown in FIGS. 3A and 3B, a first large lumen 22 and a second small lumen 23. The large and small lumens (22 and 23) may be concentric as in FIG. 3B, but may equally be separate and parallel as in FIG. 3A. Adjacent its distal end 24 pernasal intubation tube 21 is provided with an inflatable cuff 25. Small lumen 23 leads to an aperture 26 near the distal end 24 which is located within inflatable cuff 25. Large lumen 22 is closed at its distal end but communicates with a series of orifices 27 in the wall of tube 21. Orifices 27 are located between the inflatable cuff 25 and a proximal plug 28 which has an axial bore for snugly receiving the tube 21 in liquid tight manner. Proximal plug 28 is sized and shaped so as to fit in a person's nostril and to provide a substantially liquid tight seal therein. Tube 21 further has a proximal end 29 which is provided with an appropriate connection device shown in FIG. 6 in communication with the large lumen 22 by an infusion bag (not shown) containing a cold liquid, such as a cold isotonic saline solution, can be connected to the large lumen 22. In addition tube 21 has a side branch 30 which leads to the small lumen 23 and in one embodiment is terminated with the connection device 60 shown in FIG. 6, which provides an adapter 62 for connection to a source of an inflation fluid, such as air, oxygen, or of a liquid, such as a cold saline solution. In the embodiment shown in FIG. 4 an inflation bulb similar to that used as part of a mechanical sphygmomanometer is connected to adapter 62 for supply of air through small lumen 23 for inflating the inflatable cuff 25. The inflation bulb 40 is connected to a valve 41, which is opened or close in order to control inflation.

It is also envisaged that cuff 25 can alternatively be inflated using liquid under pressure. Hence it can be arranged for connection via an inlet tap or valve to a diffusion bag filled, for example, with a cold saline solution, the appropriate pressure being generated by elevating the bag to an appropriate height above the patient's head. FIG. 5 shows a suitable saline dispenser 42, having a similar valve 41 to the inflation bulb 40 of FIG. 4. The saline dispenser may be a flexible polymer bag as shown in the Figure, which may be attached to a line and raised in order to generate sufficient fluid pressure.

FIG. 2 shows a section through the left side of the person's face with another item of the illustrated apparatus, namely a venting device 31, in position for venting liquid from the left nasal cavity 32. Venting device 31 comprises a plug 33 which, like proximal plug 28, is sized and shaped so as to fit in a person's nostril and to provide a substantially liquid tight seal therein. Plug 33 has a passage 34 which provides communication for liquid therethrough from the left nasal cavity 32 through a valve 35 to a discharge tube 36 which has a proximal end 37. As illustrated, plug 33 has been inserted in left nostril 38.

Although the apparatus has been illustrated in the accompanying semi-diagrammatic drawings with the pernasal intubation tube 21 inserted through the patient's right nostril 3 (as shown in FIG. 1) and with the liquid venting device 31 inserted in the patient's left nostril 38 (as shown in FIG. 2), it will be readily appreciated by the skilled reader that it does not matter which through which nostril the pernasal intubation tube 21 is inserted. Thus the pernasal intubation tube 21 can alternatively be inserted through the patient's left nostril 38 while the venting device 31 is inserted in the patient's right nostril 3.

In use of the illustrated apparatus for First Aid treatment of a patient in danger of suffering brain damage after a stroke (such use being of course only one example of usages of apparatus according to the present invention), the patient will typically be lying, or made to lie, prone on his or her back or side. Having ensured that the patient is in an appropriate position, a paramedic or other medically qualified practitioner then inserts the pernasal intubation tube 21 through one of the patient's nostrils, for example, through the patient's right nostril 3, until its closed distal end 24 lies just below the patient's soft palate 5. The proximal plug 28 provides an approximate indication as to when this has been achieved since it will engage in the patient's nostril 3 or 38, as the case may be, and prevent further insertion of the tube 21. The paramedic can then check visually through the patient's mouth 8 to confirm that the distal end 24 is in the correct position. If desired, tube 21 can be sized so as to be slidable in proximal plug 28 while still permitting formation of a substantially liquid fluid seal; such an arrangement permits the length of tube 21 between the proximal plug 21 and distal end 24 to be varied. Next the inflatable cuff 25 is inflated using, for example, an inflation bulb until inflatable cuff 25 occludes the nasopharynx 17. It will be noted that the oropharynx 19 remains substantially unobstructed so that the patient can continue to breathe through his or her mouth 8 either unaided or with the help of a respirator (not shown). The proximal plug 28 is then inserted in the right nostril 3 to provide a substantially liquid tight seal.

A typical inflation pressure for inflating cuff 25 is about 100 mb gauge (about 1.45 psig).

The plug 33 of liquid venting device 31 is inserted in the patient's other nostril, for example, the left nostril 38, and the proximal end 37 of discharge tube 36 is connected to a suitable liquid receptacle, for example, an empty diffusion bag (not shown).

Temperature sensors can be positioned in the patient's auditory canals or attached to the patient's neck or other areas of the head or body in order to monitor the patient's brain temperature. In addition the body temperature of the patient can be monitored with, for example, the aid of a rectal thermometer.

In a First Aid situation, a diffusion bag filled with a supply of cold coolant liquid, such as a cold saline solution, at a temperature of, for example, +4° C. to +10° C. is then connected to the proximal end 29 of pernasal intubation tube 21 and elevated to a suitable height above the patient's head such that liquid can flow through the large lumen 22 and out of orifices 27 into the right or left nasal cavity 4 or 32, depending on which nostril is plugged with proximal plug 28. Upon opening valve 35, liquid will continue to flow through large lumen 22 and orifices 27 until it fills and irrigates both nasal cavities 4 and 32, thereby cooling directly the lining of the nasal cavities 4 and 32 and also the blood in the subjacent arteries and veins. Valve 35 can then either be closed or left slightly open, thereby to provide intermittent or continuous bathing of the nasal cavities 4 and 32. If the supply of cold coolant should cease, for example, by reason of the supply diffusion bag emptying, then valve 35 is closed, if it has been left open, while a fresh diffusion bag containing further cold coolant liquid is fitted to the proximal end 29 of pernasal intubation tube 21 and elevated to an appropriate height above the patient's head. Valve 35 can then be reopened.

If continuous irrigation is adopted, then a suitable flow rate for the cold saline solution is from about 25 ml/min to about 1000 ml/min.

Although the paramedic or other appropriately trained person can select whichever nostril he or she prefers through which to insert the pernasal intubation tube 21, it is preferable, if the patient is lying on his or her side, to insert it through the lowermost nostril. In this way the nasal cavities fill with cold coolant liquid from beneath.

It will be appreciated by those skilled in the art that the illustrated apparatus is simple and readily portable. It is thus well suited for use by paramedics or by persons with appropriate training in first aid, such as police officers, who are likely to be first on the scene when a person suffers a stroke in the course of his or her usual occupations. Insertion of the pernasal intubation tube 21 through one of the patient's nostrils 3 or 38 can be achieved quickly and simply without the need for any additional probe or special intubation device. Moreover it can readily be checked visually whether the distal end 24 is in the correct position behind the patient's soft palate 5 prior to inflation of cuff 25. Similarly insertion of the liquid venting device 31 in the patient's other nostril 38 or 3 is likewise quick and simple. The illustrated apparatus can be put into operation extremely speedily, which is a very important consideration in treatment of persons who have undergone a stroke and are at very great risk of suffering brain damage within a short period of time unless preventative measures are very speedily taken. Moreover it is simple to check visually that the distal end 24 is in the correct position before the cuff 25 is inflated.

It will be noted that the various forms of apparatus and the methods described herein effect only localised lowering of temperature of the head region in order to cause cooling of the brain, rather than cooling of the entire body.

In a hospital context it may be desirable to connect the proximal end 29 of pernasal intubation tube 21 to the proximal end 37 of discharge tube 36 by a cooling coil placed in a temperature-controlled refrigerant bath and to circulate cold coolant liquid continuously or intermittently for irrigation of a patient's nasal cavities to effect brain cooling.

If desired, an electric pump designed to produce and maintain a predetermined pressure may be used in place of an inflation bulb.

The temperature of the cold coolant liquid is preferably less than ambient temperature, for example, from about −4° C. to about +15° C., preferably from about +4° C. to about +10° C.

In the course of treatment of a patient to effect brain cooling, it may be desirable that the patient's brain temperature is reduced to a temperature of not less than about +30° C., preferably not less than about +32° C., e.g. about +32° C. to about +35° C. Such reduced brain temperature can be maintained, for example, for a period of a few hours up to several days or even months. A typical period of treatment may involve maintaining such a reduced brain temperature for from about 24 hours to about 72 hours. After receiving First Aid to reduce the brain temperature in the event of a stroke or other trauma that could lead to brain damage, the patient can undergo further suitable treatment in a hospital environment to lower his or her body temperature, in addition to reducing his or her brain temperature by the method of the invention.

The materials of construction of the apparatus of the invention can be selected from among those conventionally used in the field of medical intubation devices. For example, pernasal intubation tube 21 can be made from a pliable semi-rigid, soft plastics material such as a medical grade of polyethylene, polypropylene, polyvinylchloride, or the like. If desired, the distal end of pernasal intubation tube 21 may be coloured, e.g. blue or green, so as to increase its visibility and make it easier for a paramedic or other appropriately trained person to check that it is correctly in position before cuff 25 is inflated. The distal tip 24 may also include a radiopaque material to facilitate its visualisation in X-ray photographs.

Inflatable cuff 25 can be made from any material conventionally used for manufacture of cuffs on intubation devices, such as a medical grade of a silicone rubber. The pernasal intubation tube 21 of FIG. 1 is shown in more detail in FIG. 3C and has a single inflatable cuff 25 adjacent its distal end 24 which is sealed or otherwise secured in airtight fashion to the tube 21 both proximally and distally with respect to the aperture 26. FIGS. 3D and 3E show in deflated and inflated states respectively an alternative embodiment in which the tube 21 has several apertures 26 to allow passage of the inflation fluid into the cuff 25. Alternatively, as shown in FIG. 3G cuff 25 can be replaced by a balloon positioned at the distal end 24, being sealed or secured thereto in liquid tight fashion by a single line of sealing or the like proximally adjacent the distal end 24; in this case small lumen 23 can lead to an open distal end 24 and aperture 26 is not then required. In the particular example shown in FIG. 3G, the thickness of the tube tapers so that its distal end may be inflated, thus providing an inflatable occluder in the form of an integrally formed balloon 25.

It is also envisaged that, in the inflatable occluder shown in FIG. 3F, a second cuff 25B can be provided on pernasal intubation tube 21 to assist in occluding the nasopharynx, this second cuff 25B being positioned proximally with respect to the first cuff 25A but distally with respect to the aperture 27 and the small lumen 23 having further apertures (similar to apertures 26) for inflation of the second cuff. Alternatively the pernasal intubation tube 21 can be provided with a third lumen for inflating such a second cuff.

Proximal plug 28 and plug 33 are preferably made from appropriate plastics materials, such as a medical grade of polyethylene, polypropylene, or the like. If desired, proximal plug 28 and/or plug 33 can be replaced by a corresponding inflatable cuff.

Instead of using a valve 35, a tap could be used in place thereof.

Pernasal intubation tube 21 has been described as having a second small lumen 23. In an alternative construction small lumen 23 and side branch 30 are replaced by a separate tube disposed substantially parallel with the tube 21 which is connected to the inflatable cuff 25 or, in the alternative construction described above, to the balloon.

The saline solution can be an isotonic, hypertonic or hypotonic saline solution. Ringer's lactate solution, water, or any other suitable physiologically tolerated liquid can be used in place of a saline solution.

As indicated above the invention further contemplates a kit for cooling the brain of a patient in danger of suffering brain damage comprising apparatus, such as that illustrated in FIGS. 1 to 3, in combination with a source of a cold biologically acceptable liquid and a store for storing the source of cold biologically acceptable liquid and for maintaining same at a temperature suitable for effecting cooling of a patient's brain. The store can be, for example, a refrigerator or insulated storage box containing a number of diffusion bags filled with a cold saline solution at a temperature of from about +4° C. to about +10° C. Such a refrigerator or insulated storage box can be a stand alone item or can be installed, for example, in an ambulance, a doctor's vehicle, a paramedic's vehicle, or a police vehicle. A kit of this type may include plugs of different sizes for use with patients of different ages from babies to adults as the proximal plug 28 and/or the plug 33 so as to be suitable for use with nostrils of different sizes.

1. An apparatus for cooling the brain comprising:
a pernasal intubation tube having a proximal portion, a distal end for insertion through one of the nostrils of a patient requiring brain cooling, and a first lumen extending from the proximal portion to a closed distal lumen end;
an inflatable occluder provided on a distal portion of the tube for inflation by the admission of fluid to occlude the nasopharynx of the patient behind the soft palate of the patient thereby to prevent passage of liquid between the nasal cavities and the mouth of the patient;
a nostril closure arranged to cooperate with the tube for closing the one nostril of the patient and preventing fluid escaping from the nasal cavity with which the one nostril communicates;
at least one orifice in communication with the first lumen and disposed in a wall of the tube between the nostril closure and the inflatable occluder for introduction of a liquid coolant from the first lumen of the tube into the nasal cavities; and
a liquid vent for insertion in the other nostril of the patient for controlling venting of liquid coolant from the nasal cavities of the patient.

2. The apparatus according to claim 1 wherein the first lumen is provided at its proximal end with a connector for connection to a source of cold coolant liquid, preferably to a diffusion bag containing a cold saline solution.

3. The apparatus according to claim 1 wherein the inflatable occluder comprises a first cuff secured in fluid tight fashion to a distal portion of the tube.

4. The apparatus according to claim 1 wherein the inflatable occluder comprises a balloon secured in fluid tight fashion to a distal portion of the tube.

5. The apparatus according to claim 4 wherein the tube is provided with a subsidiary inflatable cuff positioned proximally upstream from the balloon.

6. The apparatus according to claim 1 further comprising a second lumen terminating at its distal end at an aperture within the inflatable occluder, said second lumen preferably communicating with a side branch tube provided towards the proximal end of the tube.

7. The apparatus according to claim 6 wherein a fluid connection is provided at the proximal end of the second lumen for connection to a source or generator of pressurised fluid.

8. The apparatus according to claim 1 wherein the nostril closure comprises a plug having a bore for receipt of the tube.

9. The apparatus according to claim 1 wherein the liquid vent comprises a plug for insertion in the other nostril having a bore providing a passage for liquid between the nasal cavity with which the other nostril communicates and a liquid outlet for controlling outflow of liquid from that nasal cavity.

10. A method of cooling the brain of a patient in danger of suffering brain damage comprising occluding the air passage leading from the nasal cavities to the mouth cavity of the patient behind the soft palate of the patient and irrigating the nasal cavities of the patient with a coolant liquid.

11. A method of cooling the brain of a patient suffering from a condition which could lead to brain damage comprises the steps of:
providing a pernasal intubator comprising a tube having a proximal portion, a distal end, a first lumen extending between the proximal portion and a closed distal lumen end, an inflatable occluder on a distal portion of the tube, a nostril closure arranged to cooperate with the tube for closing the one nostril of the patient and preventing fluid escaping from the nasal cavity with which the one nostril communicates and an orifice in communication with the first lumen and disposed in a wall of the tube between the nostril closure and the inflatable occluder;
introducing the distal end of the tube through one of the nostrils of the patient until the inflatable occluder lies in the nasopharynx behind the soft palate of the patient;
inflating the inflatable occluder so as to occlude the nasopharynx of the patient in a region behind the soft palate of the patient, thereby to prevent passage of liquid between the nasal cavities and the mouth of the patient;
inserting into the other nostril of the patient a liquid vent for controlling venting of liquid from the nasal cavities of the patient;
introducing a coolant liquid into the nasal cavities through the first lumen of the tube and the orifice so as to bathe the lining of the nasal cavities with coolant liquid; and
venting liquid from the nasal cavities of the patient through the liquid vent.

12. The method according to claim 11 wherein the step of inflating the inflatable occluder comprises supplying air under pressure through a second lumen in the tube.

13. The method according to claim 11 wherein the cooling liquid is supplied from a diffusion bag containing a charge of a cold biologically acceptable liquid.

14. The method according to claim 14 wherein the biologically acceptable liquid is a cold saline solution, preferably isotonic.

15. The method according to claim 11 wherein liquid is vented from the nasal cavities into a diffusion bag.

16. The method according to claim 11 wherein the coolant liquid is supplied at a temperature of from about +4° C. to about +10° C.

17. The method according to claim 11 wherein the coolant liquid is introduced at a rate of from about 25 ml/min to about 1000 ml/min.

18. The method according to claim 11 wherein the temperature of the brain is monitored and wherein the coolant liquid is introduced at a rate sufficient to maintain the temperature of the brain at between about 32° C. and about 35 C.

19. An apparatus for cooling the brain comprising:
pernasal intubation means comprising a tube having a proximal portion, a distal end for insertion through one of the nostrils of a patient requiring brain cooling, and a first lumen extending from the proximal portion to a closed distal lumen end;
inflatable occluding means provided on a distal portion of the tube for inflation to occlude the nasopharynx of the patient behind the soft palate of the patient thereby to prevent passage of liquid between the nasal cavities and the mouth of the patient;
fluid admission means for admission of fluid to the inflatable occluding means to effect inflation thereof so as to cause occlusion of the nasopharynx of the patient behind the soft palate of the patient;
nostril closure means arranged to cooperate with the tube for closing the one nostril of the patient and preventing fluid escaping from the nasal cavity with which the one nostril communicates;
orifice means in communication with the first lumen and disposed in a wall of the tube between the nostril closure means and the inflatable occluding means for introduction of a liquid coolant from the first lumen of the tube into the nasal cavities; and
liquid venting means for insertion in the other nostril of the patient for controlling venting of liquid coolant from the nasal cavities of the patient.

20. A kit for cooling the brain of a patient in danger of suffering brain damage comprising:
an apparatus in accordance with claim 1;
a source of a cold biologically acceptable liquid; and
a store for storing the source of cold biologically acceptable liquid and for maintaining same at a temperature suitable for effecting cooling of a patient's brain.

21. The apparatus according to claim 3 wherein the tube is provided with a subsidiary inflatable cuff positioned proximally upstream from the first cuff.

22. A kit for cooling the brain of a patient in danger of suffering brain damage comprising:
an apparatus in accordance with claim 19;
a source of a cold biologically acceptable liquid; and
a store for storing the source of cold biologically acceptable liquid and for maintaining same at a temperature suitable for effecting cooling of a patient's brain.
